(19) United States
(12) Patent Application Publication
(10) Pub. No.: US 2024/0343859 A1
(43) Pub. Date: Oct. 17, 2024

(54) POLY(ETHYLENE BRASSYLATE-CO-DIOXANONE) COPOLYMERS, METHOD OF SYNTHESIS AND BIOMEDICAL DEVICES MADE THEREFROM

(71) Applicants: University of Rhode Island Board of Trustees, Kingston, RI (US); Yale University, New Haven, CT (US)

(72) Inventors: Matthew Kiesewetter, North Kingstown, RI (US); Kassie Picard, Kingston, RI (US); Jinal Pothupitiya, New Haven, CT (US); W. Mark Saltzman, New Haven, CT (US)

(73) Assignees: University of Rhode Island Board of Trustees, Kingston, RI (US); Yale University, New Haven, CT (US)

(21) Appl. No.: 18/628,340

(22) Filed: Apr. 5, 2024

Related U.S. Application Data
(63) Continuation of application No. PCT/US2022/045681, filed on Oct. 4, 2022.
(60) Provisional application No. 63/252,957, filed on Oct. 6, 2021.

(51) Int. Cl. C08G 63/08; A61L 27/18; A61L 27/54; A61L 27/58

(52) U.S. Cl. CPC C08G 63/08; A61L 27/18; A61L 27/54; A61L 27/58

(57) ABSTRACT

Embodiments of the present invention comprises one or more of an article composed of a copolymer, a copolymer made from a process, and a process for the preparation of a copolymer composition by metal free enzyme ring-opening polymerization of a monomer composition comprising i) an ethylene brassylate monomer; ii) a 1-4 dioxan-2-one (DO); iii) lipase B from Candida antarctica (Novozyme® 435) and at an elevated temperature, processing the monomer reactants i) and ii) to a copolymer via ring-opening polymerization under solvent-free conditions and a nitrogen atmosphere in the absence of a metal and the presence of the lipase to produce a random EB-co-DO copolymer.

### STATEMENT REGARDING FEDERALLY SPONSORED RESEARCH OR DEVELOPMENT

This invention was made with government support under grant number HL139756 awarded by the National Institutes of Health, grant number 1554830 awarded by the National Science Foundation and grant number AID-OAA-A-10-00060 awarded by the USAID. The government has certain rights in the invention.

### CROSS-REFERENCE TO RELATED APPLICATIONS

This application is a continuation of International Application No. PCT/US2022/045681 entitled “POLY (ETHYLENE BRASSYLATE-CO-DIOXANONE) COPOLYMERS, METHOD OF SYNTHESIS AND BIOMEDICAL DEVICES MADE THEREFROM”, filed Oct. 4, 2022, which claims priority to and the benefit of the filing of U.S. Provisional Patent Application No. 63/252,957 entitled “POLY (ETHYLENE BRASSYLATE-CO-DIOXANONE) COPOLYMERS, METHOD OF SYNTHESIS AND BIOMEDICAL DEVICES MADE THEREFROM”, filed on Oct. 6, 2021, and the specification and claims thereof are incorporated herein by reference.

### BACKGROUND OF THE INVENTION

### Field of the Invention (Technical Field)

Synthesis of polymers for biomedical implants can be expensive, use excess organic solvents and some use metals that must be removed prior to production of the biomedical article of manufacture. Therefore, use of a more sustainable method for the synthesis of polymers having one or morse aspects of being a less expensive method to manufacture, does not include organic solvents nor include the use of metals that must be removed prior to product can have both economic benefits and less environmental harm during manufacture of the polymer. A novel polymer, such as disclosed herein, has utility that can be used for biomedical applications ranging from nanoparticles as platforms that can be functionalized with an oligonucleotide, peptide, enzyme, compound or any combination thereof for example. In addition, the novel polymer can be used to encapsulate compounds such as drugs and/or oligonucleotides, peptides, proteins or any combination thereof or the novel polymer can be used as a coating on prosthetics are as material for surgical sutures, and/or as biodegradable platform/scaffold for cells/bone growth or as a combination drug.

Synthetic accessibility and reproducibility are two considerations in producing a biomaterial on an industrial scale. Biocompatibility, biodegradation, and effectiveness as a drug delivery agent are the practical considerations to consider for biomedical applications of a novel polymer such as disclosed here.

Providing polymer matrices that have better physical and chemical properties for biomedical implants, and which are made of sustainable materials is an aspect of the present invention. Polymers that can be derived from more sustainable sources, and still function in an appropriate and safe way are desirable.

Synthesis of polymers for biomedical implants can be expensive, use excess organic solvents thus use of more sustainable methods can have both economic benefits and less environmental harm during manufacture.

Ethylene brassylate

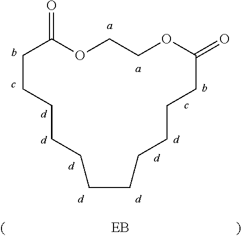 [SMILES: O=C1CCCCCCCCCCCC(=O)OCCO1]

is a 17 member ring lactone that is commercially available as a monomer. EB has been primarily used in the fragrance industry. The homopolymer of ethylene brassylate has been known to have similar mechanical properties to polycaprolactone

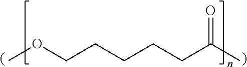

Polycaprolactone is extensively used in the biomedical industry for manufacture of biomedical devices such as sutures and other biodegradable implants. Since caprolactone

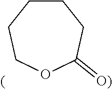

is a monomer that is derived from fossil fuel-based compounds, there is a need for a more sustainable source of starting material which is derived from plant sources and/or is not fossil fuel based.

Pentadecalactone co-p-dioxanone was synthesized by the following reaction:

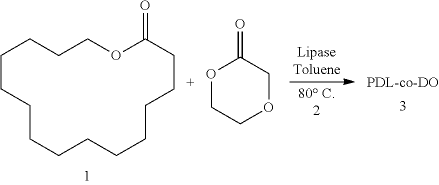

Pentadecalactone (1) and p-dioxanone (2) are reacted in the presence of toluene and lipase at about 80° C. to produce pentadecalactone-co-dioxanone copolymer (3) and/or [poly (PDL-co-DO)] which is a copolyester of an isodimorphic system, which remain semicrystalline over the whole range of compositions. This copolymer has been used for fabricating bioabsorbable sutures, orthopedic devices, and controlled drug release. This material was processed into subcutaneous implants and nanoparticles, which were found to have high rates or biocompatibility in vitro and in vivo models. Upon degradation, this material produces bioproducts that are easily excreted by the body \as it has been shown to degrade via hydrolysis. The nanoparticles synthesized from PDL-co-DO copolymers were shown to have continuous and controlled drug release profiles for pharmaceutical compositions such as the anti-cancer drug, doxorubicin, and siRNA.

In contrast to pentadecalactone, the monomer, EB, is one or more of the following: cheaper, sustainable, not fossil fuel derived and liquid at room temperature.

Ethylene brassylate (EB) is a macro (di) lactone which is a plant oil-derived dilactone. When treated with ring opening organic catalysts, a high molecular weight polyester polymer is produced. The use of organocatalysts for the polymerization of ethylene brassylate was reported in Organocatalyzed Synthesis of Aliphatic Polyesters from Ethylene Brassylate: A Cheap and Renewable Macrolactone. ACS Macro Lett. 3, 849-853 (2014). Ethylene brassylate was polymerized via a ring-opening polymerization using organocatalysts under bulk and solution conditions at 80° C. The polymerizations of EB was carried out in the presence of several organic catalysts, such as dodecylbenzenesulfonic acid (DBSA), diphenyl phosphate (DPP), p-toluenesulfonic acid (PTSA) and bases, 1,5,7-triazabicyclo [4.4.0] dec-5-ene (TBD), 1,2,3-tricyclohexylguanidine (TCHG), and 1,2,3-triisopropylguanidine (TIPG), using benzyl alcohol as initiator. The resulting poly (ethylene brassylate) aliphatic polyesters produced were characterized by NMR, SEC, MALDI-TOF, DSC, and TGA and displayed molecular weights ranging from 2 to 13 kg mol-1 and polydispersity index (PDI) between 1.5 and 2. The results of this study show that poly (ethylene brassylate) is a semicrystalline polyester similar to poly (ϵ-caprolactone) with slightly higher melting and glass transition temperatures (Tm=69° C., Tg=−33° C.) and good thermal stability.

### BRIEF DESCRIPTION OF EMBODIMENTS OF THE PRESENT INVENTION

A copolymer of ethylene brassylate-co-dioxanone is one aspect of the present invention. Another aspect of the present invention is use of the copolymer of ethylene brassylate-co-dioxanone as a biocompatible biopolymer and biobased copolymer. A copolymer of ethylene brassylate-co-dioxanone is useful in the manufacturing of biomedical devices such as pharmaceutical/drug-releasing implants owing to the copolymer's degradability, and biocompatibility. A copolymer according to one embodiment of the present invention may also be useful for one or more of the following: encapsulation of a drug/gene by the polymer matrix and controlled drug delivery/gene therapy, dental and medical implants/devices, sutures, staples, clips and meshes, orthopedic fixation devices and bio-scaffolds for tissue engineering and elastomeric films and medical device coatings.

A further aspect of the disclosed copolymer of the present invention and method of making the same is the copolymer is derived from a plant (for example Castor plant) and as such, the starting material is renewable. Another aspect of the present invention provides for a cost savings during manufacture of the copolymer composition and articles manufactured therefrom as compared to other polymer starting materials such as caprolactone and Pentadecalactone. For example in 2021, the cost of starting materials for biocompatible polymers such as Caprolactone—10 kg=$2160, Pentadecalactone—10 kg=$1474 as compared to Ethylenebrassylate—10 kg=$523 illustrates the economic advantage for a novel copolymer as disclosed herein. Further, the cost of the production of biomedical devices such as implants made with ethylene brassylate reduces the overall manufacturing cost significantly. Thus, making the biomedical devices more affordable when made of the polymeric material described herein. Further, given the EB monomer is sourced from renewable plant sources according to one embodiment of the present invention also assists in the economic and environmental benefits of embodiments of the present invention.

An ethylene brassylate (EB) copolymer, and method of production, is described according to one embodiment of the present invention. In one embodiment of the present invention, a synthesis of EB copolymeric material via the ring opening polymerization (ROP) of the macrolactone monomer, ethylene brassylate (EB), and cyclic lactone, dioxanone (DO), is described according to one embodiment of the present invention. For example, in one embodiment of the present invention, a copolymerization of EB and DO under solvent free conditions is described to provide for one or more of the following: cost effective polymer, sustainable staring reagents, and biocompatible materials formed therefrom.

The monomers, ethylene brassylate (EB) “A” and dioxanone (DO) “B”, are reacted with immobilized lipase via interfacial activation of lipase B from Candida antarctica on a resin (Novozyme® 435) at about 80° C. for about 3 hours under a nitrogen atmosphere according to one embodiment of the present invention. The polymer/copolymer [A]ₙ[B]ₘ-[A]ₙ or [B]ₘ[A]ₙ[B]ₘ where n is the number of repeating units of A (EB) such as any whole number from 1-10000 and m is the number of repeating units of B (DO) such as any whole number from 1-10000 for example (AB)ₙ or (AABB)ₙ or (AB-AABB)ₙ or any permutation thereof (Block copolymer, alternating copolymer, periodic copolymer) material formed is isolated by removing an enzyme used for ROP, for example a lipase enzyme, for example Novozyme 435 via filtration and removing unreacted starting material (ethylene brassylate and dioxanone) by precipitating out the polymer/copolymer and dissolving the monomers in methanol. It should be understood that the monomeric units may be incorporated randomly into the copolymer chain sometimes unevenly in the polymer chain. Alternatively, the monomers may be incorporated into the copolymer chain in nearly equimolar amounts. Further still, a block of one monomer is joined to a block of a second monomer.

One embodiment of the present invention relates to the development of a new polymeric biomaterial for application as one or more of the following: a biodegradable, biocompatible, drug delivery, subdermal implant made from a novel copolymer described herein.

Another embodiment of the present invention provides for the synthesis of a novel polymer as described herein.

In one embodiment, a combination of ethylene brassylate (EB) and p-dioxanone (DO) are reacted to produce poly (EB-co-DO) random copolymers in a novel process that yields a biocompatible and biodegradable drug delivery agent For example in one embodiment of the present invention, the molecular weights evaluated were between 10,000 and 150,000 g/mol.

One aspect of an embodiment of the present invention provides for polymer matrices that have similar or enhanced physical properties for biomedical devices/implants, tissue scaffolding, second skin compared to commercial devices/implants/products made of sustainable materials. In addition, polymer compositions as disclosed herein are useful for food packaging, horticultural materials and cosmetics according to an additional embodiment of the present invention. Our rationale is that polymers can be derived from more sustainable sources, and still function in an appropriate and safe way. The polymerization step of the polymer/copolymer as described herein occurs in the absence of excess organic solvent or any organic solvent and/or occurs in the absence of heavy metal catalysts. Synthesis of polymers known in the industry for biomedical devices/implants can be expensive, use excess organic solvents, and require the use of heavy metal catalysts. Therefore, use of more sustainable methods such as described here to synthesize a polymer/copolymer as described herein has both economic benefits and produces less environmental harm during the manufacturing process.

In one embodiment, Poly (ethylene brassylate-co-dioxanone) is a new polyester that is synthesized for example using enzymatic ROP. In one embodiment the enzymatic ROP of the EB-co-DO copolymer is polymerized in solvent-free and/or metal free reaction conditions, and as described herein, conditions for the synthesis process are easier, require less post synthesis purification to remove solvents and/or metals, and more environmentally friendly as the starting monomer is derived from plant based source and not derived from petroleum products.

One disadvantage in the production of polymers for biomedical applications is reproducibility of the material. The length of the polymer chains dictate many of the observable properties in the bulk material. Controlling the polymerization to create chains of a uniform size is a challenge that all polymer chemists face and this is particularly important in biomaterials, where the performance must be reliable over time and among batches. To overcome the stated disadvantage, the use of organocatalysis for the copolymerization is presented, which makes for improved control of reaction kinetics and more uniform polymer chain lengths.

The poly (ethylene brassylate-co-dioxanone) copolymer is a novel polyester that is easily synthesized via enzyme catalyzed ring-opening polymerization. The unique combination of the 17-membered macrolactone, ethylene brassylate, with the cyclic ester, p-dioxanone produces a polyester that can be further modified to form subdermal implants for example. Preliminary results suggest that this material outperforms commercially used contraceptive implants in terms of drug release profiles and ease of synthesis according to one embodiment of the present invention.

One embodiment of the present invention provides for a process for preparing a copolymer composition by enzyme ring-opening polymerization of a monomer composition. The process comprises polymerizing i) an ethylene brassylate (EB) monomer and ii) a 1-4 dioxan-2-one (DO) at an elevated temperature to form a copolymer of EB and DO via enzyme ring-opening polymerization under solvent-free conditions and a nitrogen atmosphere in the absence of a metal. For example, the enzyme is selected from a lipase, for example a Lipase B. In one embodiment, the lipase is present at between about 4 wt % to about 25 wt %. For example, the lipase is immobilized on a platform, for example a resin such as a bead. The lipase may be on the surface or within the platform or both. In a further embodiment, the ethylene brassylate monomer is present at between about 0.25 mmol and about 0.75 mmol and the DO is present at between about 0.25 mmol to about 0.75 mmol. In one embodiment, the reaction is conducted at the elevated temperature of between about 50° C. and about 120° C. Further still, the reaction does not proceed at temperatures less than about 50°° C. and greater about 150° C.

In a further embodiment of the process, the starting material i) and/or ii) are removed from the reaction and from the copolymer composition by dissolving the unreacted starting material i) and ii) in a solvent.

The polymer is for example a random copolymer. For example, the copolymer formed by the process comprises

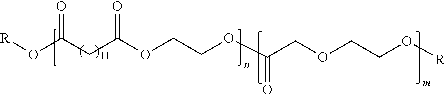

wherein R is a chain terminating species selected from methyl (CH₃) or proton (H) and wherein n is 1-10000 repeating units and m is 1-10000 repeating units or any whole number there between.

Another embodiment of the present invention comprises an article formed from the copolymer

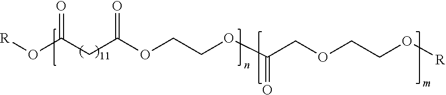

wherein R is a chain terminating species selected from methyl (CH₃) or proton (H) and wherein n is 1-10000 repeating units and m is 1-10000 repeating units or any whole number there between. For example, the article is a biocompatible article such as an implant. For example, the biocompatible article is also biodegradable. Further still an article made of the copolymer as detailed herein is combined with a pharmaceutical composition for use in or on a subject in need of treatment with the article combined with the pharmaceutical compound or composition.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIG. 1 illustrates an NMR profile from reaction scheme 1 products.

FIG. 2 illustrates an NMR profile for the reactants and product of scheme 1 reaction.

FIG. 3 illustrates the A and B peaks from scheme 1 reactants and products.

FIG. 4 illustrates an NMR profile for the reactants and products of copolymerization EB: DO of reaction scheme 2.

FIG. 5 illustrates the conversion over time of EB (circle) and DO (squares) products for no preheating of monomers for reaction scheme 2.

FIG. 6 illustrates the conversion over time of EB (squares) and DO (circles) for preheated monomers for reaction scheme 2.

FIG. 7 illustrates the percentage conversion of reactants were monitored over time for change and the result indicates that the conversion of the monomers do not change after several hours for reaction scheme 2.

FIG. 8 illustrates the percent conversion of monomer EB (circles) and DO (squares) for reaction scheme 3.

FIG. 9 illustrates the conversion of monomers EB (circles) and DO (squares) over time for reaction scheme 4.

FIG. 10 illustrates the percent conversion of EB (circles) and DO (squares) over time for reaction scheme 6.

FIG. 11 illustrates the EB (circles) and DO (squares) percent conversion over time for reaction scheme 7.

FIG. 12 illustrates the percent conversion of monomer PDL (circles) and EB (squares) over time is for reaction scheme 8.

FIG. 13 illustrates the NMR profile for EB, PEB, PDL and PPDL is provided for reaction scheme 8.

FIG. 14 illustrates mass over time for JUP-2-150 A TGA for reaction scheme 9.

FIG. 15 illustrates NMR analysis with positions 1-3 marked for reaction scheme 9.

FIG. 16 illustrates the NMR when DO is 50%, 40%, 30%, and 20% from reaction scheme 10.

FIG. 17 illustrates biomedical implant made from a PEB-co-DO created as described herein according to one embodiment of the present invention.

FIGS. 18A-E illustrates the three-point test and compression test analysis compared to commercially available sino implants and another biodegradable polymer implant.

FIGS. 19A-C illustrates the rheological properties of EB-co-DO polymer

FIGS. 20A-D illustrates the cross section of polymer implants loaded with (FIGS. 20C-D) and without (FIGS. 20A-B) drug.

FIG. 21 illustrates LNG (drug) loading capacity and drug release profile for poly (EB-co-DO) implants.

FIGS. 22A-B illustrates DSC data for poly (EB-co-DO).

FIG. 23 illustrates histology image of the skin grown over the poly (EB-co-DO) implant after two months in an in vivo model.

### DETAILED DESCRIPTION OF THE INVENTION

Embodiments of the present invention are further illustrated by the following non-limiting examples.

### EXAMPLES

Reaction scheme 1 illustrates the solvent-free reaction of 1.0 mmol of EB catalyzed with lipase B from Candida antarctica (Novozyme® 435) at 80° C. to produce EB hompolymer.

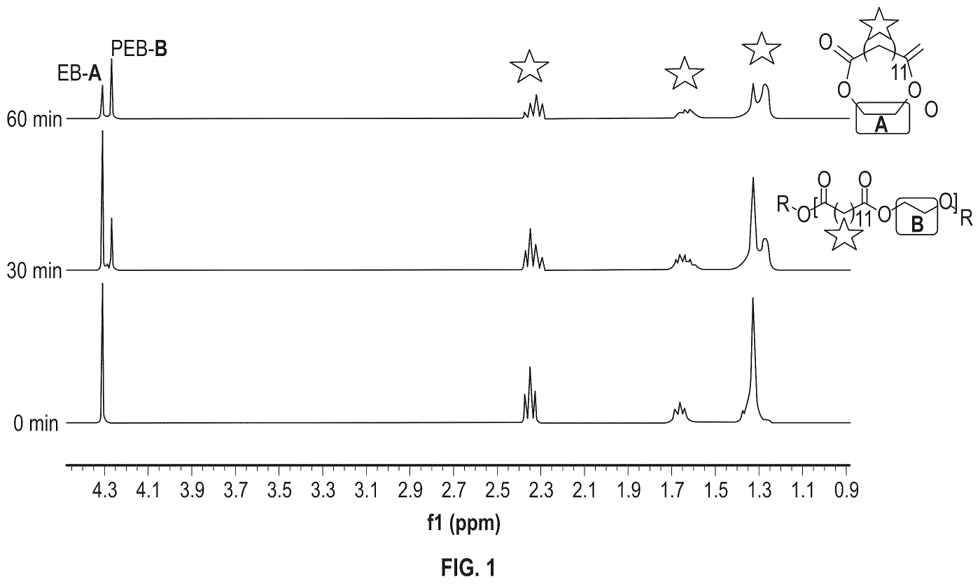

Referring now to FIG. 1, an NMR profile from reaction scheme 1 is illustrated.

Reaction scheme 1 identifies position A on the EB ring and the corresponding A position in the EB homopolymer.

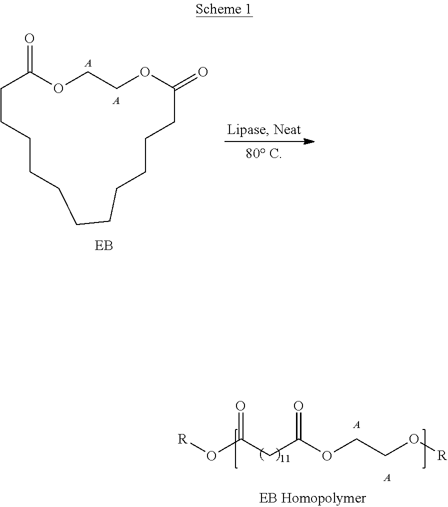

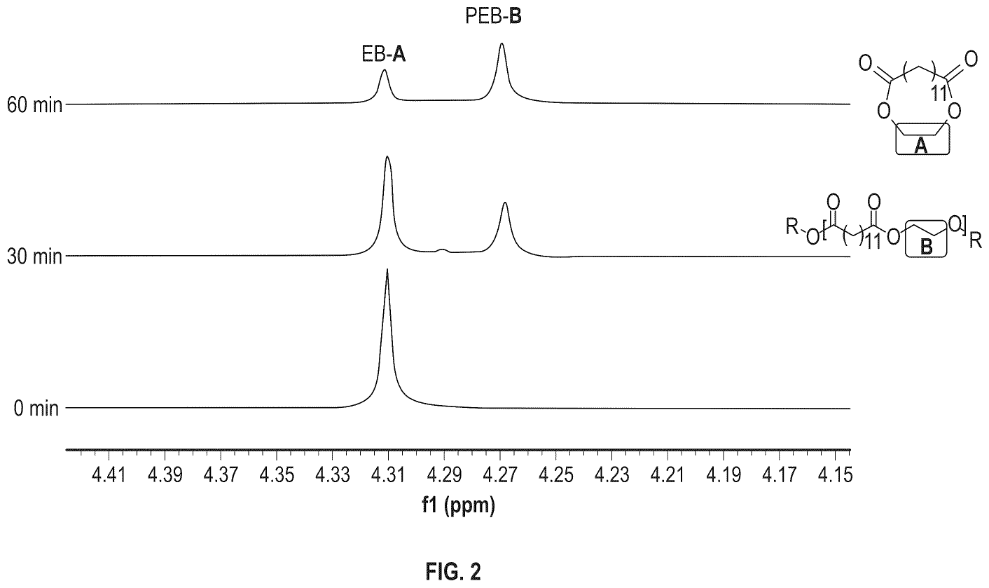

Referring now to FIG. 2, an NMR profile for the reactants EB and product PEB of reaction scheme 1 is illustrated.

Using the same conditions from scheme 1, the below (scheme 2) EB-co-DO copolymer is synthesized.

Reaction scheme 2 illustrates the solvent-free copolymerization of 0.5 mmol EB reacted with 0.5 mmol 1-4 dioxan-2-one (DO) in the presence of lipase B from Candida antarctica (Novozyme® 435) at 80° C. to produce poly (EB-co-DO).

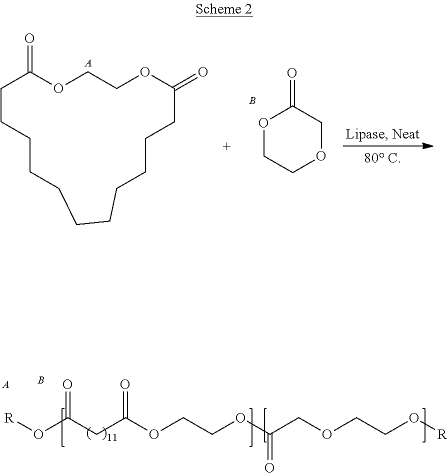

wherein the copolymer illustrated above and further in this disclosure may also be represented as copolymer

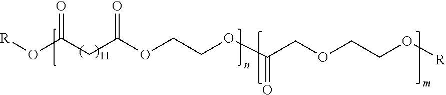

wherein n and m are repeating units from 1-10000 and/or 1-1000 and any whole number in between.

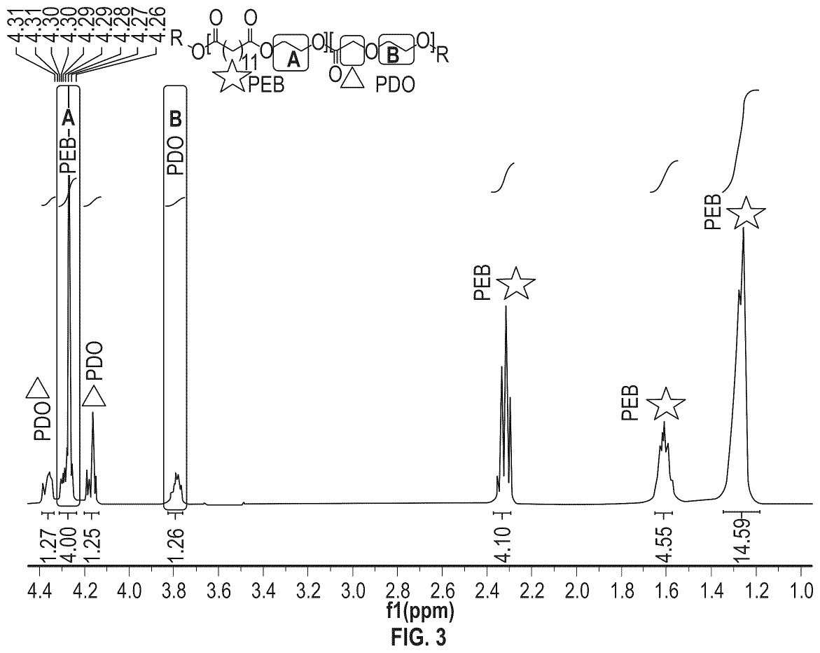

FIG. 3 illustrates the NMR profile of A and B peaks from reaction scheme 2.

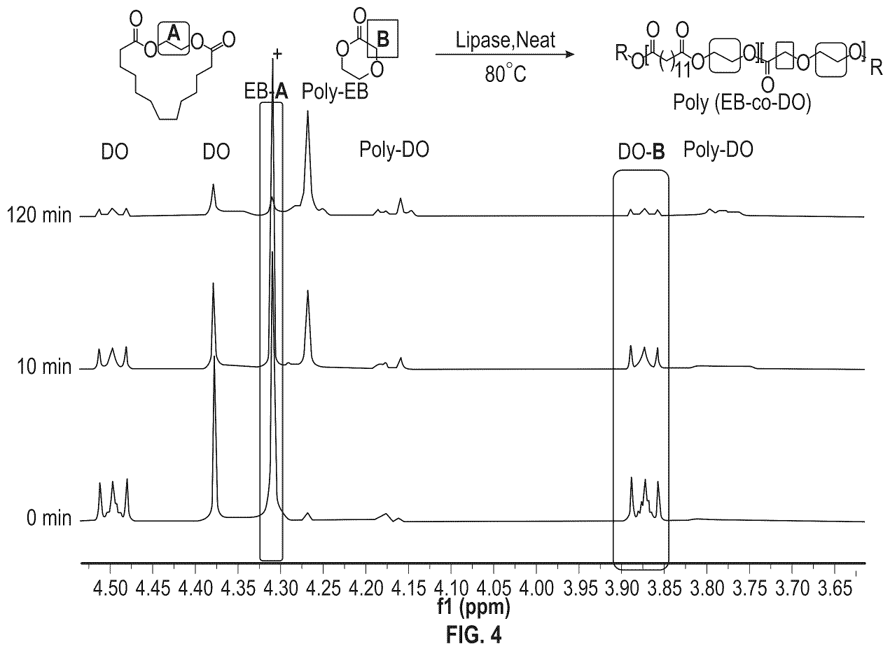

FIG. 4 illustrates the NMR profile for the reactants and products of copolymerization EB:DO reaction scheme 2.

Further, the conversion vs. time for the reaction from scheme 2 was analyzed when monomers were preheated. After 2 hours of the reaction, the percent (%) conversion was measured as follows in Table 1.

TABLE 1

| Reactant | % conversion | ratio |
|---|---|---|
| DO | 85 | / |
| EB | 66 | / |
| EB:DO | / | 56:44 |

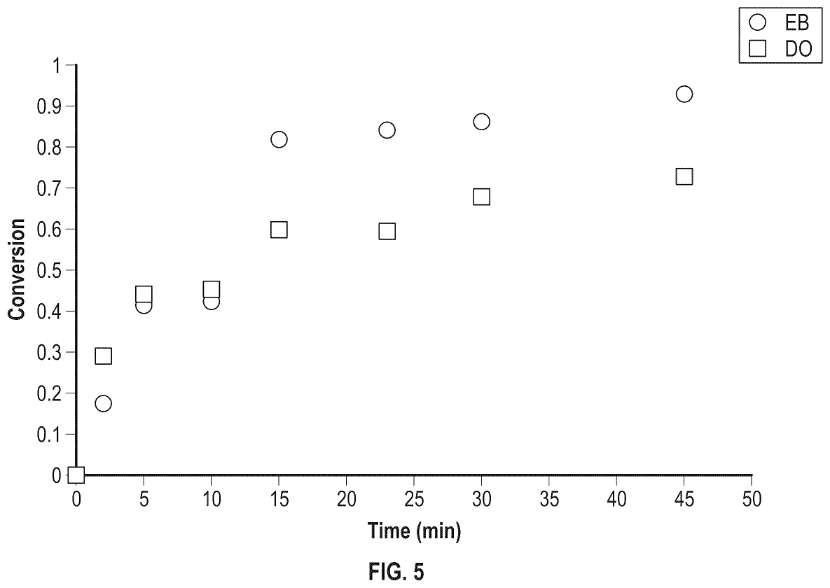

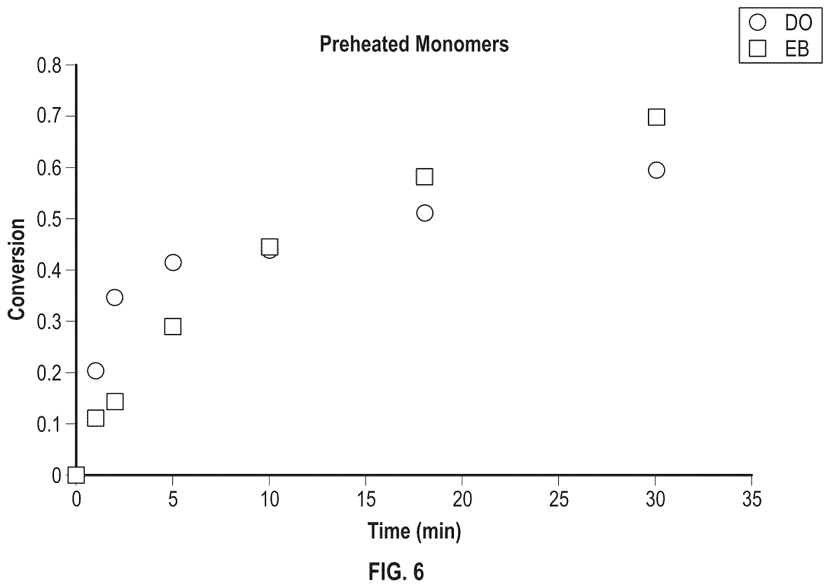

FIG. 5-6 illustrates the conversion of monomer EB and DO over time is shown for no preheating of monomers and for preheated monomers from reaction scheme 2.

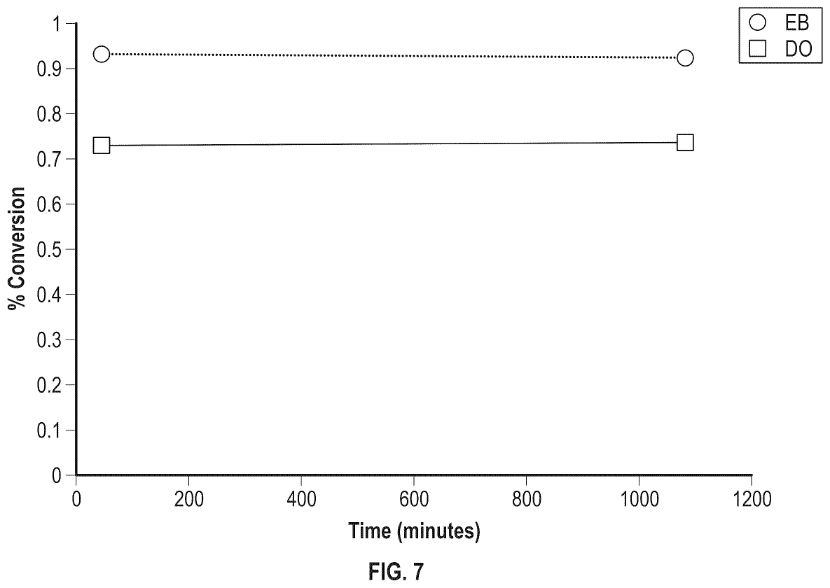

FIG. 7 illustrates the percentage conversion of EB and DO vs. time after overnight heating of the reaction from scheme 2. The plateau in conversion indicates that the conversion of the monomers does not change after several hours.

TABLE 2 below summarizes the separate reactions based upon the feed ratio of EB: DO. The table summarizes the unit ratio of each monomer in the polymer backbone as well as Mn, Mw, and PDI, which are all important factors that determine characteristics of the material.

TABLE 2

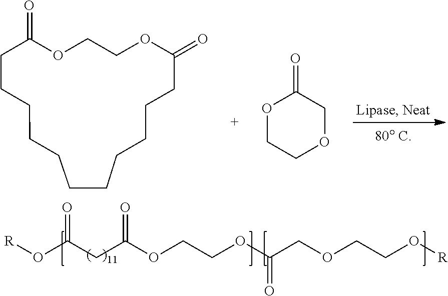

| | EB:DO Feed^{a} | EB:DO | mmol % | | | | Retention |
|---|---|---|---|---|---|---|---|
| Entry | (mol/mol) | Unit^{b} | enzyme | Mn^{c} | Mw^{c} | PDI | Time (min) |
| 1 | 90:10 | 91:9 | 16.7% | 30000 | 117000 | 3.9 | 16.68 |
| 2 | 80:20 | 86:14 | 16.7% | 39000 | 119000 | 2.0 | 16.72 |
| 3 | 70:30 | 75:25 | 16.7% | 46000 | 105000 | 2.3 | 16.75 |
| 4 | 60:40 | 66:34 | 16.7% | 29000 | 81000 | 2.8 | 17.77 |
| 5 | 50:50 | 58:42 | 16.7% | 29000 | 71000 | 2.4 | 17.94 |
| 6 | 40:60 | $1:49 | 16.7% | 30000 | 52000 | 1.8 | 18.10 |
| 7 | 50:50 | 55:45 | 33.4% | 27000 | 49000 | 1.8 | 18.03 |
| 8 | 50:50 | 62:38 | 11.4% | 31000 | 66000 | 2.1 | 17.71 |
| 9 | 50:50 | 64:36 | 4.2% | 25000 | 72000 | 2.9 | 17.70 |

^{a}Total amount of monomer sums to 2 mmol.
^{b}Monomer incorporation determined by ¹H NMR.
^{c}Determined by GPC (CH₂Cl₂) vs polystyrene standards.

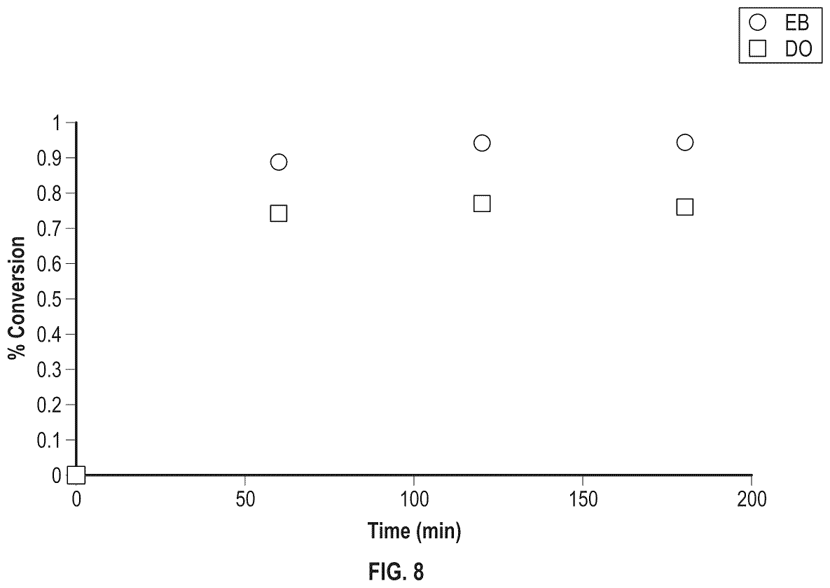

Reaction scheme 3 indicates that 0.5 mmol of EB was reacted with 0.5 mmol of 1-4 dioxan-2-one (DO) in the presence of 17% weight lipase, neat at 80°° C. to produce poly (EB-co-DO). The retention time, weight average molecular weight “Mw”, number average molecular weight “Mn” and Mw/Mn or polydispersity index “PDI” is provided for this reaction in Table 3. The percent conversion of Monomer EB and DO is provided in FIG. 8.

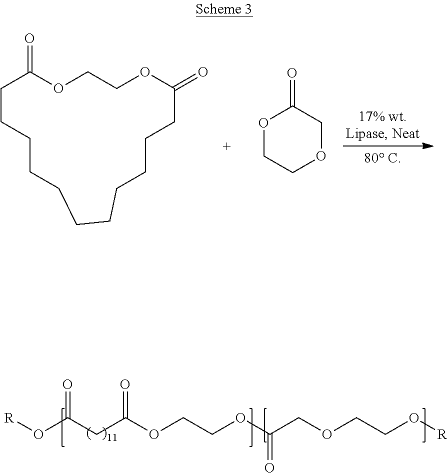

TABLE 3

| Retention Time | Mw | Mn | PDI |
|---|---|---|---|
| 18.06 min | 47000 | 24000 | 1.958 |

Reaction scheme 4 reacts 0.5 mmol and 0.5 mmol 1, 4 dioxan-2-one in the presence of 5% weight Lipase, neat at 80° C.

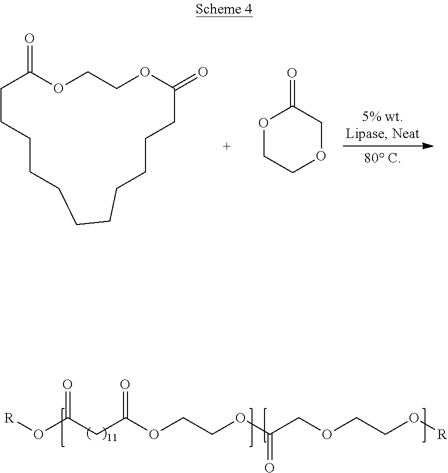

Table 4 provides for the retention time, Mw, Mn and PDI for the copolymerization from scheme 4.

TABLE 4

| Retention Time | Mw | Mn | PDI |
|---|---|---|---|
| 17.63 min | 75000 | 23000 | 3.211 |

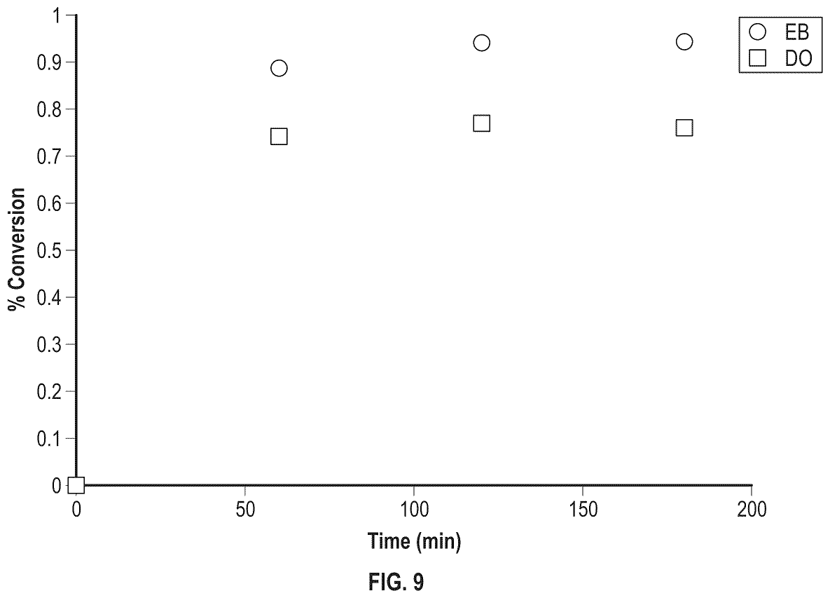

The conversion of monomers EB and DO in reaction scheme 4 over time is provided in FIG. 9.

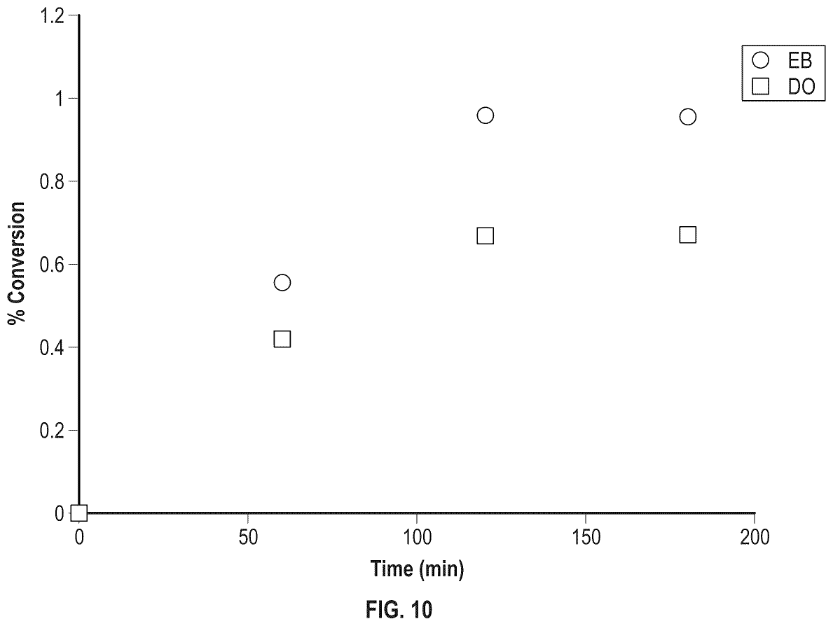

Reaction scheme 6 shows 0.75 mmol of EB reacting with 0.25 mmol 1,4-dioxan-2-one (DO) in the presence of 8% weight lipase, neat at 80° C. to produce EB-DO copolymer. Table 6 provides the retention time, Mw, Mn and PDI for this reaction. The percent conversion of EB and DO over time is provided in FIG. 10.

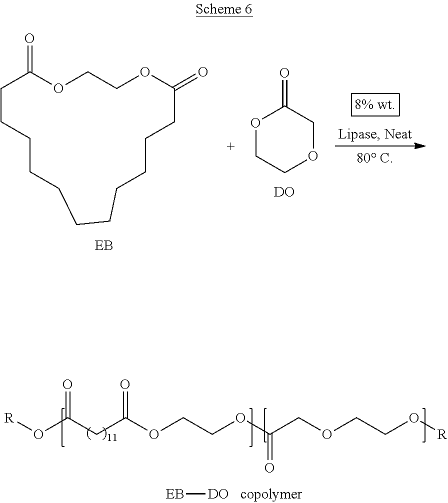

TABLE 6

| Retention Time | Mw | Mn | PDI |
|---|---|---|---|
| 16.83 min | 104284 | 43132 | 2.418 |

Reaction scheme 7 is the reaction of 0.25 mmol EB with 0.75 mmol of 1,4 dioxan-2-one in the presence of 14% weight lipase, neat at 80° C. to produce EB-DO copolymer.

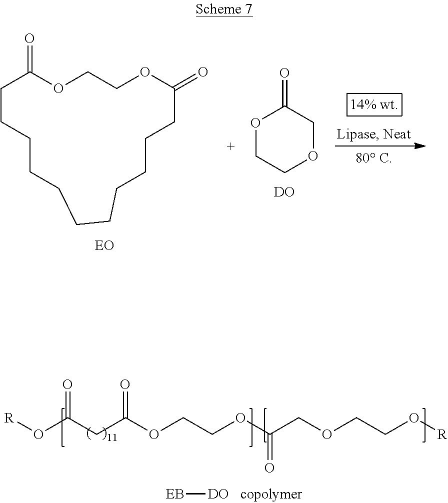

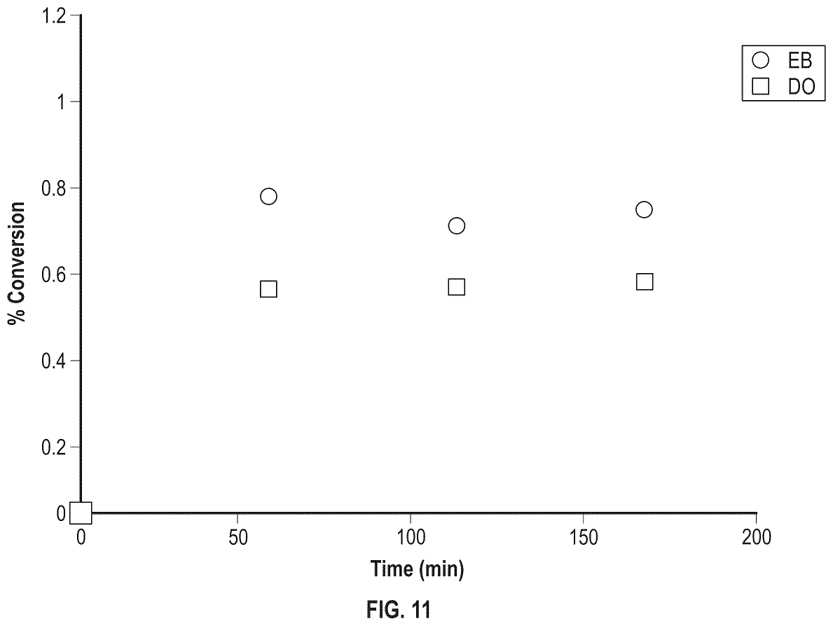

Table 7 illustrates the retention time, Mw, Mn, and PDI for the reaction in scheme 7. FIG. 11 provides the EB and DO percent conversion over time.

TABLE 7

| Retention Time | Mw | Mn | PDI |
|---|---|---|---|
| 18.98 | 31531 | 17589 | 1.793 |

Reaction scheme 8 shows the reaction of EB with oxacyclohexadexadecan-2-one (PDL) in the presence of lipase, neat at about 80° C. for about 150 min to produce EB-PDL copolymer.

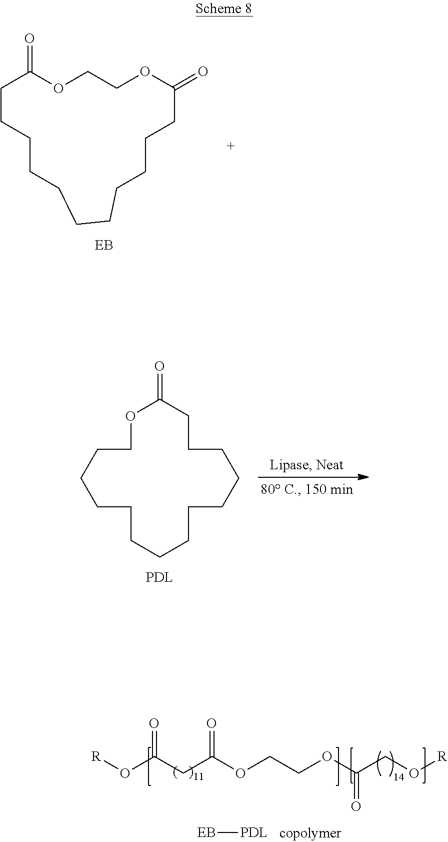

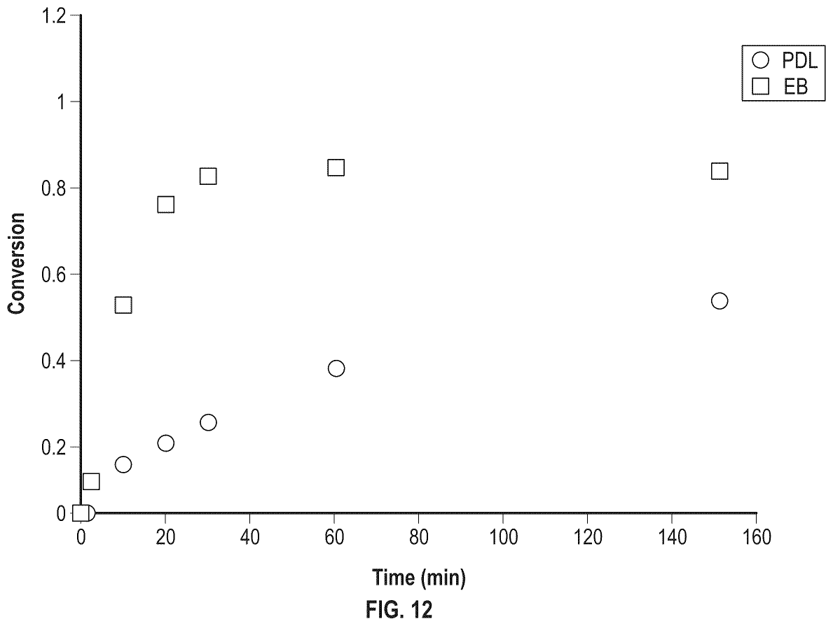

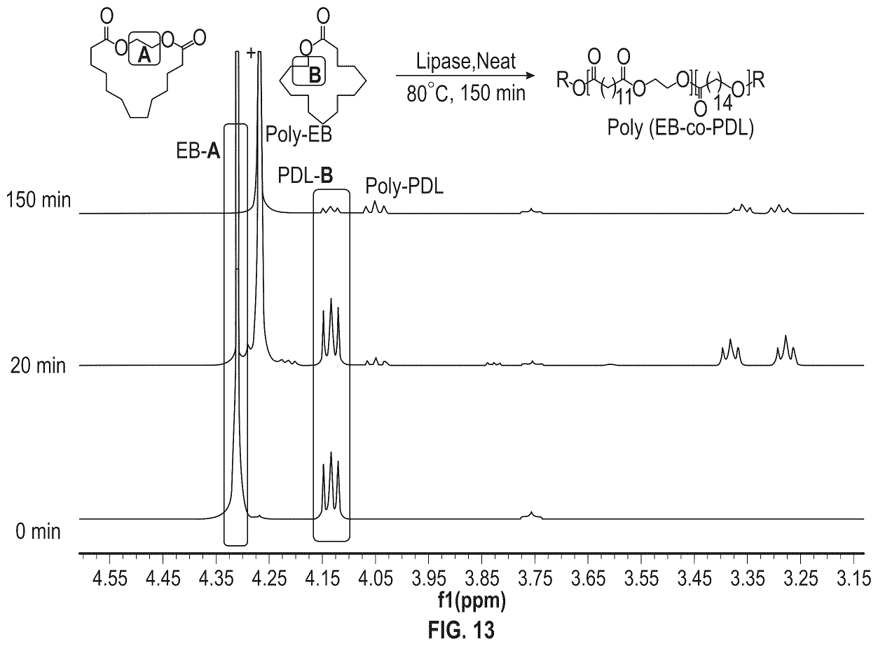

The ratio of PDL:EB is 28:72, Mw is 50000, Mn is 75000 and the PDI is about 1.501. The percent conversion of monomer PDL and EB over time is provided for in FIG. 12. The NMR profile for EB, PEB, PDL and PPDL is provided in FIG. 13.

Reaction scheme 9 provides for the reaction of EB and glycolide at a feed ration of 1:1 (mole) in solvent-free lipase at about 80° C. to produce a copolymer wherein Mn is 44,000 g/mol, PDI is 1.7.

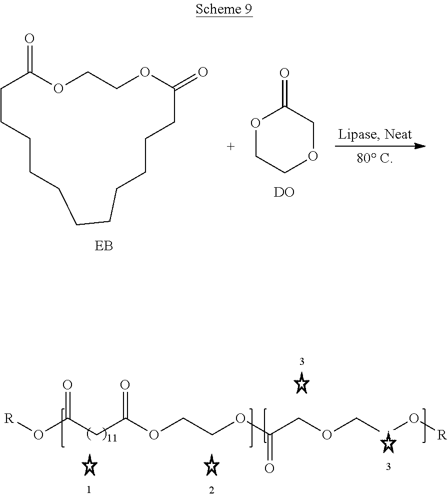

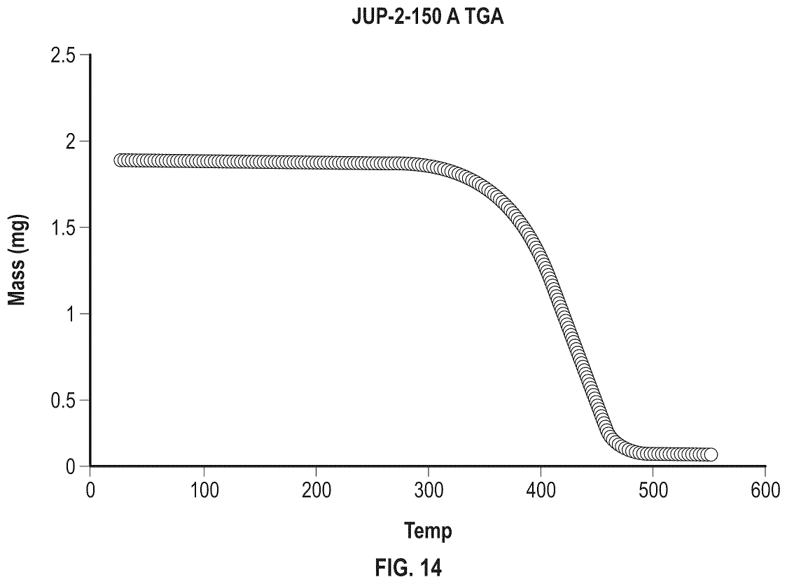

FIG. 14 illustrates mass over time for JUP-2-150 A TGA.

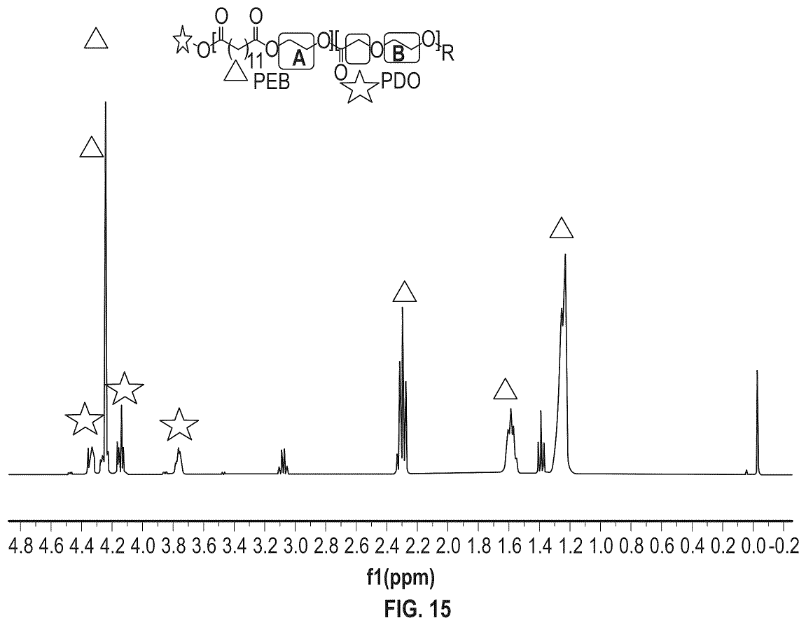

FIG. 15 illustrates NMR of JUP-2-150A (EB-co-DO) polymer with positions within the NMR marked triangle and star wherein triangle at f1 (ppm) between about 2.3-1.2 corresponds to position 1 (C₁-C₁₁) from reaction scheme 9 and the triangle at f1 (ppm) of about 4.2 corresponds to position 2 of reaction scheme 9 and wherein the star at f1 (ppm) of between about 4.3 and 3.8 corresponds to position 3 from reaction scheme 9.

Reaction scheme 10 illustrates a reaction wherein EB is varied in relation to dioxonone for the reaction in the presence of solvent-free Lipase at about 80° C. to produce EB-co-DO polymer.

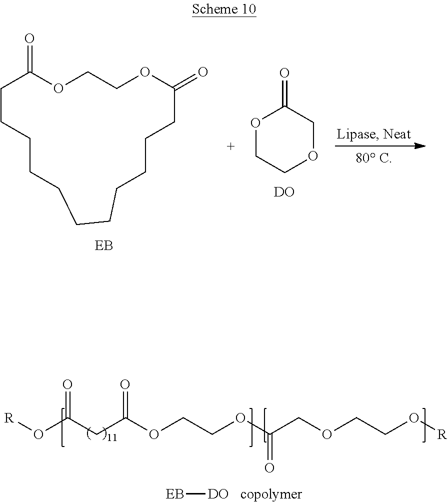

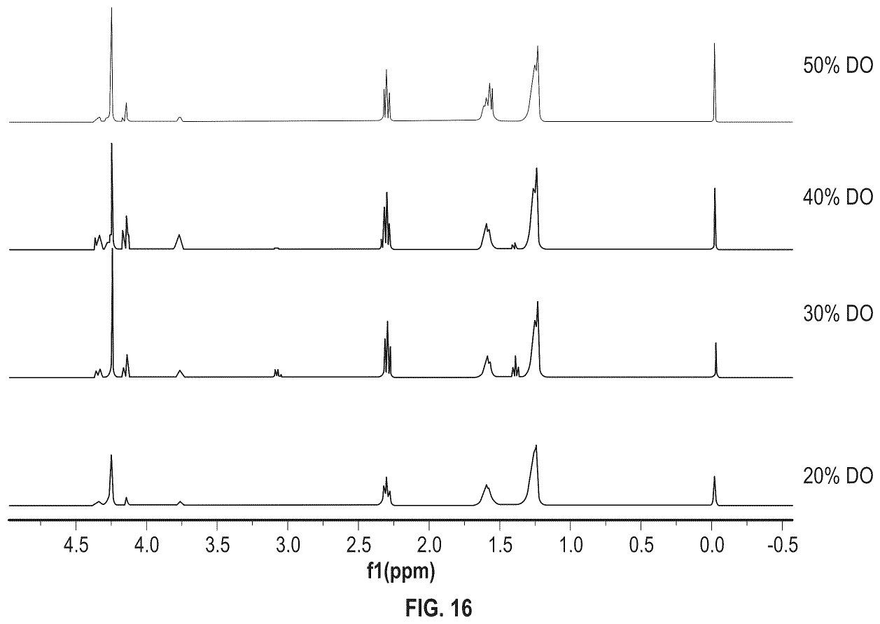

FIG. 16 illustrates the NMR when percent DO is varied to 50%, 40%, 30%, and 20%.

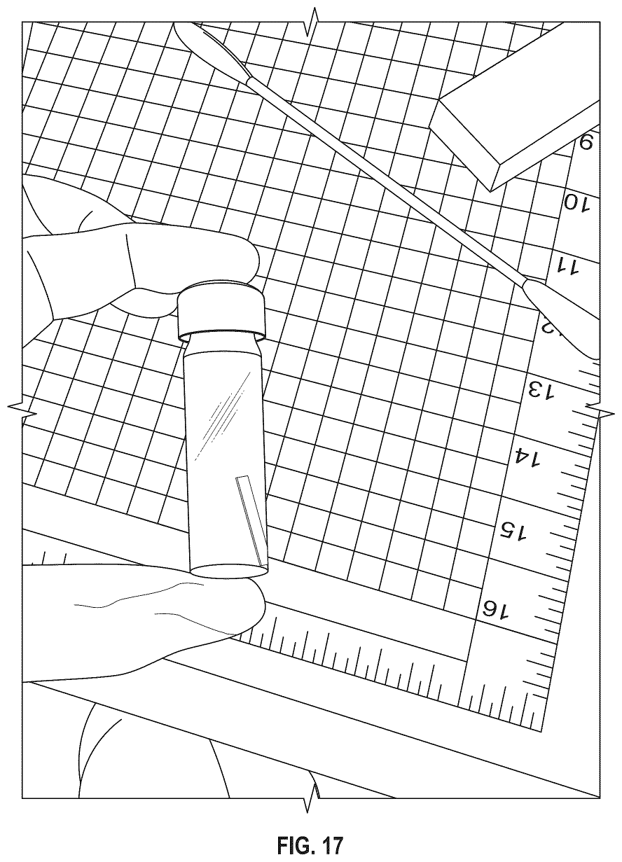

FIG. 17 illustrates an image of a biomedical implant made from a poly EB-co-DO polymer as described herein. Implants are made by weighing the required mass of polymer and filling into a Teflon mold. The polymer in the mold is melted at 80° C. and pressed into shape while polymer is cooling down slowly to room temperature. The implants are made to weigh between 50-100 mg, 2 cm in length and 2 mm in diameter. For mice implants the 2 cm implants were cut to smaller implants which weigh about 20 mm. In one embodiment, the synthesis of Poly (EB-co-DO) proceeds under solvent-free and metal free reaction conditions. The reaction is catalyzed by lipase enzyme (Novozyme 435) under a nitrogen atmosphere at 80° C.

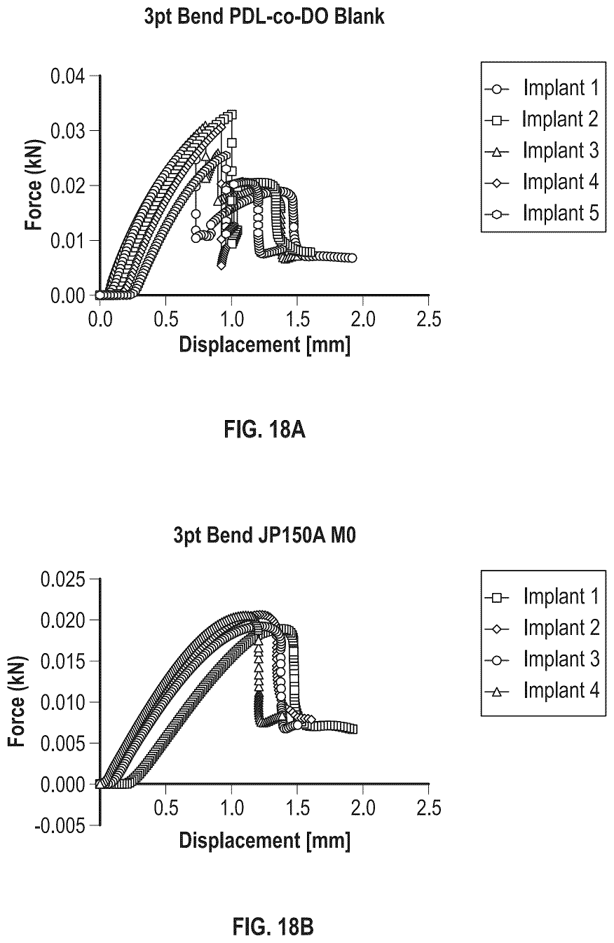

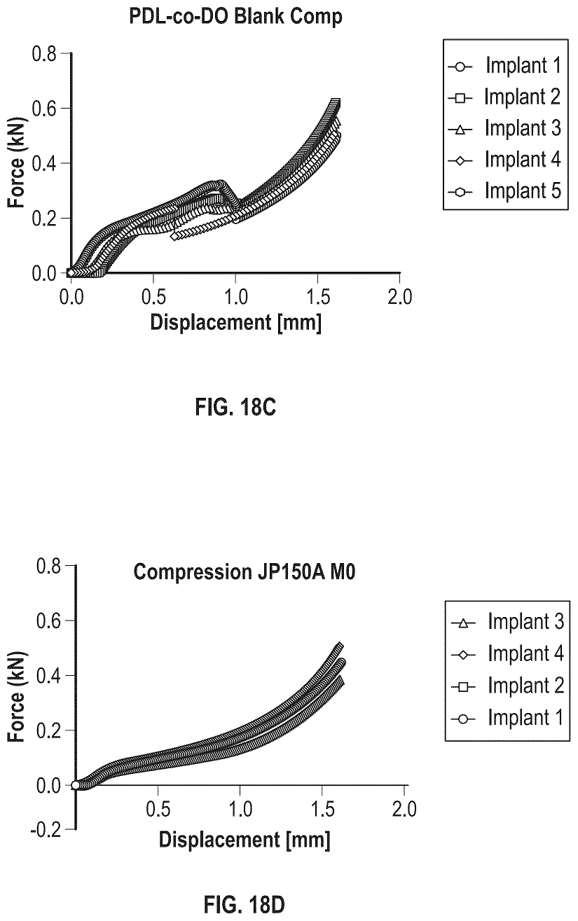

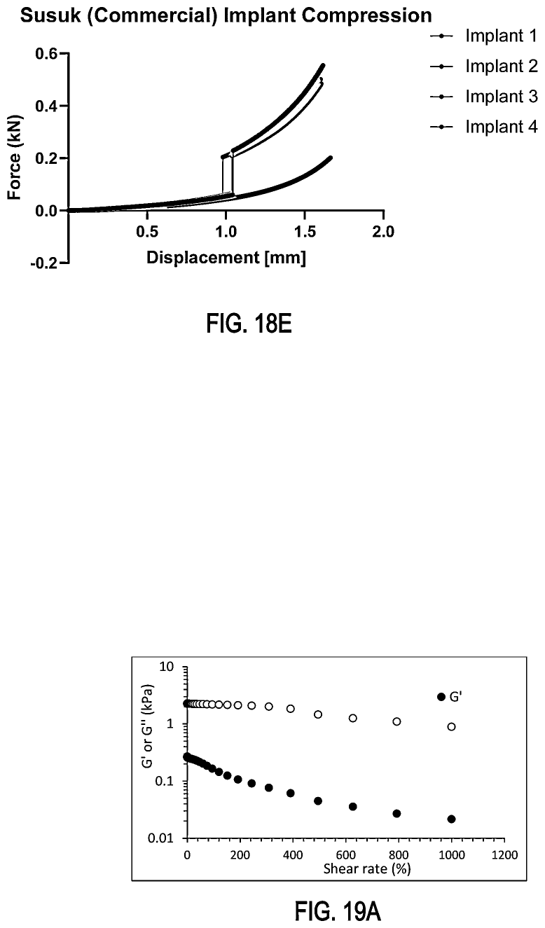

FIGS. 18A-E depicts the mechanical testing conducted on the previously studied material, three point bending test poly (PDL-co-DO blank) (A), three point bending test of the novel material, poly (EB-co-DO referred to as JP150A) (B), compression analysis of poly (PDL-co-DO blank) implant (C), compression analysis of poly (EB-co-DO referred to as JP150A) implant (D), compression analysis of commercially available sino implant €.

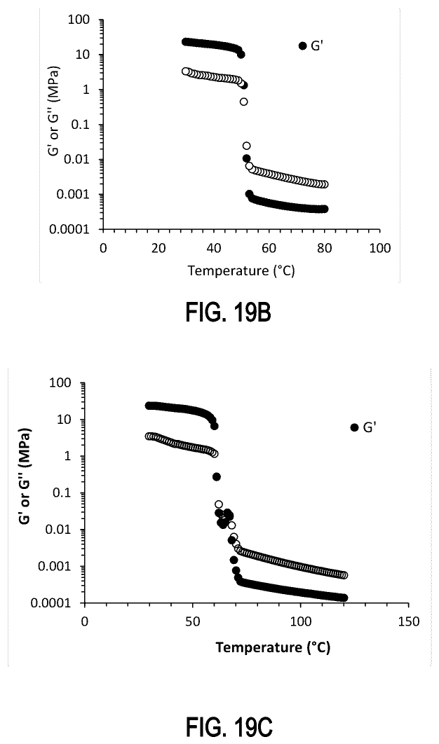

FIGS. 19A-C shows the rheological studies conducted on an embodiment of a novel EB-co-DO polymer.

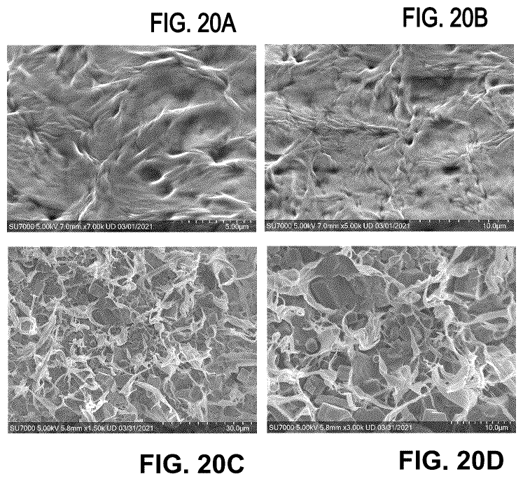

FIGS. 20A-D illustrates scanning electron microscope images of the novel material with drug loaded (FIGS. 20C-D) and without drug loaded (FIGD. 20A-B).

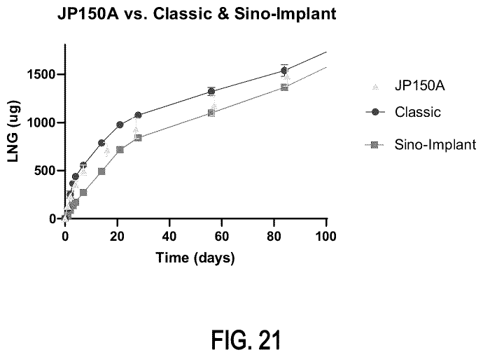

FIG. 21 illustrates LNG (drug) loading capacity and drug release profile for poly (EB-co-DO) implants. The drug release profiles for the previously studied material, poly (PDL-co-DO) (classic), the novel material, poly (EB-co-DO) (triangle), commercially available commercially sino implant (square).

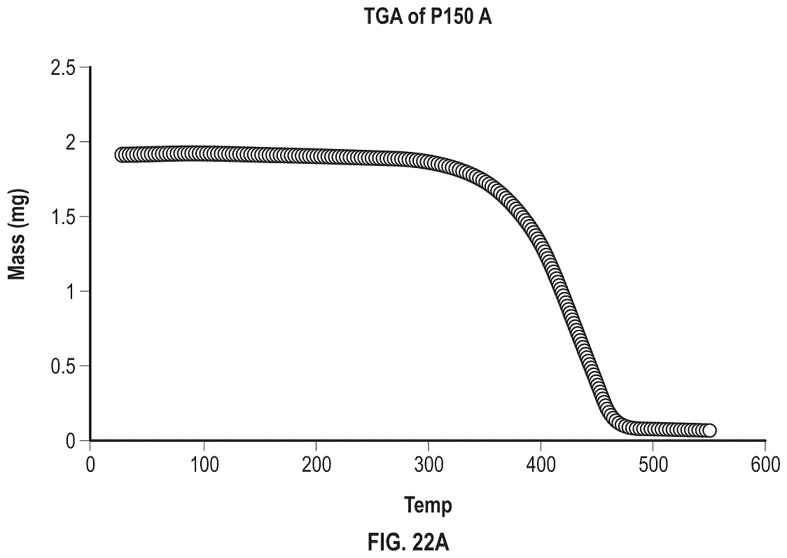

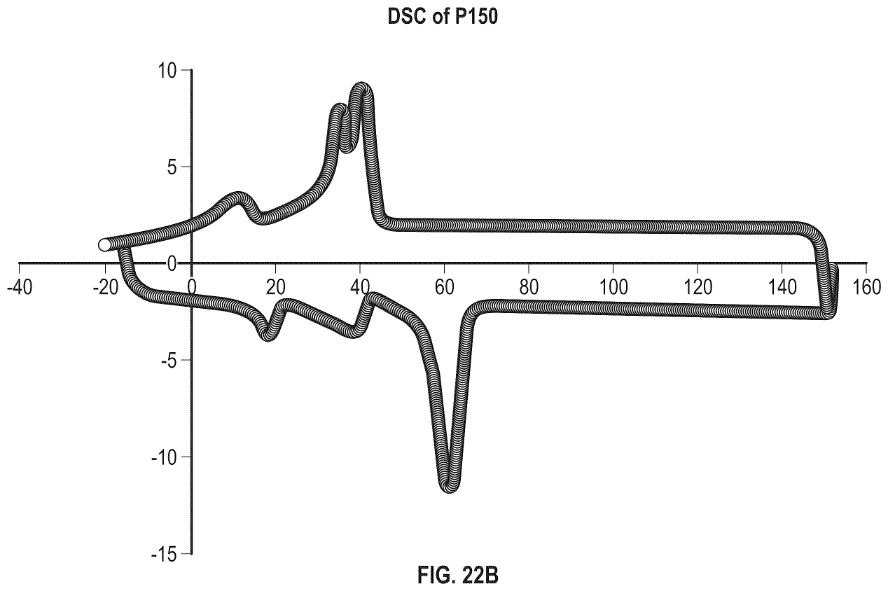

FIGS. 22A-B illustrates the bTGA and DSC of P150A over a range of

temperatures.

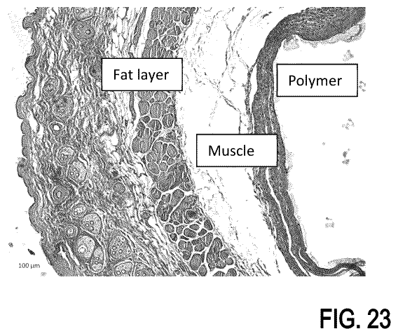

FIG. 23 illustrates histology images of the skin grown over the poly (EB-co-DO) implant after two months in an in vivo model.

In at least one embodiment, and as readily understood by one of ordinary skill in the art, the apparatus according to the invention will include a general or specific purpose computer or distributed system programmed with computer software implementing the steps described above, which computer software may be in any appropriate computer language, including C++, FORTRAN, BASIC, Java, assembly language, microcode, distributed programming languages, etc. The apparatus may also include a plurality of such computers/distributed systems (e.g., connected over the Internet and/or one or more intranets) in a variety of hardware implementations. For example, data processing can be performed by an appropriately programmed microprocessor, computing cloud, Application Specific Integrated Circuit (ASIC), Field Programmable Gate Array (FPGA), or the like, in conjunction with appropriate memory, network, and bus elements.

Embodiments of the present invention provide a technology-based solution that overcomes existing problems with the current state of the art in a technical way to satisfy an existing problem for biodegradable implants and manufacturers thereof. The process for manufacture can be automated and controlled by an algorithm run on a processor. Embodiments of the present invention achieve important benefits over the current state of the art, such as increased flexibility, decreased cost, improved processing, solvent free, metal free reactions and starting with renewable monomer that is plant derived.

The preceding examples can be repeated with similar success by substituting the generically or specifically described reactants and/or operating conditions of this invention for those used in the preceding examples.

Note that in the specification and claims, “about” or “approximately” means within twenty percent (20%) of the numerical amount cited.

As used herein “a”, “an”, “said” or “the” means one or more unless otherwise indicated.

The term “biocompatible” as used herein, generally refers to materials that are, along with any metabolites or degradation products thereof, generally non-toxic to the recipient, and do not cause any significant adverse effects to the recipient. Generally speaking biocompatible materials are materials which do not elicit a significant inflammatory or immune response when administered to a patient.

The term “biodegradable” as used herein, generally refers to a material that will degrade or erode under physiologic conditions to smaller units or chemical species that are capable of being metabolized, eliminated, or excreted by the subject. The degradation time is a function of composition and morphology. Degradation times can be from hours to years. In one embodiment the degradation time is tuned based upon one or more of the following: polymer composition and/or molecular weight of the polymer and/or formation.

The term “copolymer” as used herein, generally refers to a single polymeric material that is comprised of two or more different monomers. The copolymer can be of any form, such as random, block, graft, etc. The copolymers can have any end-group, including capped or acid end groups.

The term “implant”, as used herein, generally refers to a device that is inserted into the body.

The term “nanoparticle”, as used herein, generally refers to a particle having a diameter, such as an average diameter, from about 10 nm up to but not including about 1 micron, preferably from 100 nm to about 1 micron. The particle can have any shape. Nanoparticles having a spherical shape are generally referred to as “nanospheres”.

All computer software disclosed herein may be embodied on any computer-readable medium (including combinations of mediums), including without limitation CD-ROMs, DVD-ROMs, hard drives (local or network storage device), USB keys, other removable drives, ROM, and firmware.

Although the invention has been described in detail with particular reference to these embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference.

What is claimed is:

1. A process for preparing a copolymer composition by enzyme ring-opening polymerization of a monomer composition comprising:
i) an ethylene brassylate (EB) monomer;
ii) a 1-4 dioxan-2-one (DO); and
at an elevated temperature, polymerizing the monomer composition i) and ii) to a copolymer of EB and DO via enzyme ring-opening polymerization under solvent-free conditions and a nitrogen atmosphere in the absence of a metal and the presence of a lipase.

2. The process of claim 1 wherein the ethylene brassylate monomer is present at between about 0.25 mmol and about 0.75 mmol and the DO is present at between about 0.25 mmol to about 0.75 mmol.

3. The process of claim 1 wherein the lipase is present at between about 4 wt % to about 25 wt %.

4. The process of claim 1 wherein the lipase is Lipase B.

5. The process of claim 1 wherein the reaction is conducted at the elevated temperature of between about 50° C. and about 120° C.

6. The process of claim 1 wherein the reaction does not proceed at temperatures less than about 50° C. and greater about 150° C.

7. The process of claim 1 wherein the lipase is immobilized on a platform.

8. The process of claim 1 wherein the platform is a resin.

9. The process of claim 1 wherein the resin is a bead.

10. The process of claim 1 further comprising:
removing an unreacted starting material i) and/or ii) from the copolymer composition by dissolving the unreacted starting material i) and ii) in a solvent.

11. The process of claim 1 wherein the copolymer is a random copolymer.

12. A copolymer obtainable by a process of claim 1 having the structure comprising: 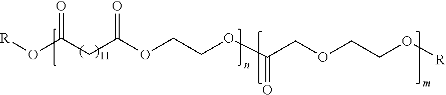
wherein R is a chain terminating species selected from methyl (CH₃) or proton (H) and wherein n is 1-10000 repeating units and m is 1-10000 repeating units.

13. An article comprising the copolymer of claim 12.

14. The article of claim 13 wherein the article is a biocompatible implant.

15. The article of claim 14 wherein the biocompatible implant is biodegradable.

16. The article of claim 13 wherein a pharmaceutical composition is combined with the article.

17. The article of claim 13 wherein the article is selected from a suture, staple, or clip.

18. The article of claim 13 wherein the article is a bio-scaffold.

19. The article of claim 13 wherein the article is a film.

20. The article of claim 13 wherein the article is a mesh.
